(19) United States
(12) Patent Application Publication
(10) Pub. No.: US 2014/0276334 A1
(43) Pub. Date: Sep. 18, 2014

(54) FLUID DRAIN TUBE WITH CONNECTOR

(71) Applicant: BeckerSmith Medical, Inc., Los Angeles, CA (US)

(72) Inventor: Jan M. Eckermann, Los Angeles, CA (US)

(73) Assignee: BeckerSmith Medical, Inc., Los Angeles, CA (US)

(21) Appl. No.: 14/209,983

(22) Filed: Mar. 13, 2014

Related U.S. Application Data
(60) Provisional application No. 61/779,793, filed on Mar. 13, 2013.

(51) Int. Cl. A61M 27/00; A61M 25/02

(52) U.S. Cl. CPC A61M 27/006; A61M 25/02
USPC 604/8

(57) ABSTRACT

A device for signaling the imminent removal of a tube from an opening in a patient's skin. The device includes a tube attachment mechanism that is configured to be temporarily secured to a tube, a tether having a proximal end and a distal end, and a patient attachment mechanism attached to the distal end of the tether. The proximal end of the tether is secured to the tube attachment mechanism.

### CROSS REFERENCE TO RELATED APPLICATIONS

This application claims the benefit of U.S. Provisional Application No. 61/779,793 filed Mar. 13, 2013, which is incorporated herein by reference in its entirety.

### FIELD OF THE INVENTION

The present invention relates to a fluid drain tube associated with a device for connecting the tube to the user of the tube, and more particularly to a cerebrospinal fluid (“CSF”) drain tube with a clip that signals the patient when the tube is being pulled.

### BACKGROUND OF THE INVENTION

It is common for patients with tubes or catheters inserted into their skull or other body portion (e.g., a vein) for fluid drainage or the like to pull out the tube or move about in a way that causes the tube to begin to move out of the skull/body portion. U.S. Pat. Nos. 4,707,906, 6,247,211 and 3,990,454 are all incorporated by reference in their entireties.

### SUMMARY OF THE PREFERRED EMBODIMENTS

In accordance with a first aspect of the present invention there is provided a device for signaling the imminent removal of a tube from an opening in a patient's skin. The device includes a tube attachment mechanism that is configured to be temporarily secured to a tube, a tether having a proximal end and a distal end, and a patient attachment mechanism attached to the distal end of the tether. The proximal end of the tether is secured to the tube attachment mechanism. In a preferred embodiment, the patient attachment mechanism is a clip that is configured to be clipped to a patient's hair. In another preferred embodiment, the patient attachment mechanism is an adhesive patch configured to be adhered to a patient's skin. In another preferred embodiment, the device includes a plurality of tethers each having a proximal end and a distal end, with the proximal end of each tether being secured to the tube attachment mechanism, and a patient attachment mechanism attached to the distal end of each of the tethers. In an embodiment, each of the patient attachment mechanisms is a clip that is configured to be clipped to a patient's hair. In another embodiment, each of the patient attachment mechanisms is an adhesive patch configured to be adhered to a patient's skin.

In a preferred embodiment, the device includes a tube and the tube attachment mechanism is permanently attached to the tube. In another embodiment, the tube and the device are provided in a kit.

In accordance with another aspect of the present invention there is provided a method that includes the steps of obtaining a tube and obtaining a device for signaling the imminent removal of the tube from an opening in a patient's skin. The device includes a tube attachment mechanism, a tether having a proximal end secured to the tube attachment mechanism and a distal end, and a patient attachment mechanism attached to the distal end of the tether. The method further includes the steps of securing the tube attachment mechanism to the tube, forming an opening in a patient's skin, inserting the tube into the opening, and securing the patient attachment mechanism to the patient. The steps can be performed in any order. For example, the tube attachment mechanism can be secured to the tube before or after the tube is inserted into the opening in the patient's skin. Also, the patient attachment mechanism can be secured to the patient before or after insertion of the tube.

In a preferred embodiment, the patient attachment mechanism is secured to hair on the patient's head. In another preferred embodiment, the patient attachment mechanism is secured to the patient's skin. In an embodiment, the device includes a plurality of tethers each having a proximal end and a distal end. The proximal end of each tether is secured to the tube attachment mechanism, and a patient attachment mechanism is attached to the distal end of each of the tethers. The method further includes the step of securing each of the patient attachment mechanisms to the patient. In a preferred embodiment, the tube is inserted into an opening in the patient's skin and an opening in the patient's skull.

The invention, together with additional features and advantages thereof, may be best understood by reference to the following description.

### BRIEF DESCRIPTION OF THE DRAWINGS

FIG. 1 is a perspective view of a signaling device attached to a patient's hair and to a tube inserted into the patient's skull in accordance with a preferred embodiment of the present invention,

FIG. 2 is a perspective view of a signaling device attached to a patient's skin and to a tube inserted into the patient's skull in accordance with a preferred embodiment of the present invention;

FIG. 3 is an elevational view of the device of FIG. 1;

FIG. 4 is a perspective view of a signaling device attached to a patient's hair and to a tube inserted into the patient's skull in accordance with another preferred embodiment of the present invention;

FIG. 5 is a perspective view of a signaling device attached to a patient's hair and to a tube inserted into the patient's skull in accordance with another preferred embodiment of the present invention; and

FIG. 6 is a perspective view of a signaling device attached to a patient's skin and to a tube inserted into the patient's skull in accordance with another preferred embodiment of the present invention.

Like numerals refer to like parts throughout the several views of the drawings.

### DETAILED DESCRIPTION OF THE PREFERRED EMBODIMENTS

The following description and drawings are illustrative and are not to be construed as limiting. Numerous specific details are described to provide a thorough understanding of the disclosure. However, in certain instances, well-known or conventional details are not described in order to avoid obscuring the description. References to one or an other embodiment in the present disclosure can be, but not necessarily are, references to the same embodiment; and, such references mean at least one of the embodiments.

Reference in this specification to “one embodiment” or “an embodiment” means that a particular feature, structure, or characteristic described in connection with the embodiment is included in at least one embodiment of the disclosure. Appearances of the phrase “in one embodiment” in various places in the specification do not necessarily refer to the same embodiment, nor are separate or alternative embodiments mutually exclusive of other embodiments. Moreover, various features are described which may be exhibited by some embodiments and not by others. Similarly, various requirements are described which may be requirements for some embodiments but not other embodiments.

The terms used in this specification generally have their ordinary meanings in the art, within the context of the disclosure, and in the specific context where each term is used. Certain terms that are used to describe the disclosure are discussed below, or elsewhere in the specification, to provide additional guidance to the practitioner regarding the description of the disclosure. For convenience, certain terms may be highlighted, for example using italics and/or quotation marks: The use of highlighting has no influence on the scope and meaning of a term; the scope and meaning of a term is the same, in the same context, whether or not it is highlighted. It will be appreciated that the same thing can be said in more than one way.

Consequently, alternative language and synonyms may be used for any one or more of the terms discussed herein. Nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and is not intended to further limit the scope and meaning of the disclosure or of any exemplified term. Likewise, the disclosure is not limited to various embodiments given in this specification.

Without intent to further limit the scope of the disclosure, examples of instruments, apparatus, methods and their related results according to the embodiments of the present disclosure are given below. Note that titles or subtitles may be used in the examples for convenience of a reader, which in no way should limit the scope of the disclosure. Unless otherwise defined, all technical and scientific terms used herein have the same meaning as commonly understood by one of ordinary skill in the art to which this disclosure pertains. In the case of conflict, the present document, including definitions, will control.

It will be appreciated that terms such as “front,” “back,” “top,” “bottom,” “side,” “short,” “long,” “up,” “down,” and “below” used herein are merely for ease of description and refer to the orientation of the components as shown in the figures. It should be understood that any orientation of the components described herein is within the scope of the present invention.

As shown in FIGS. 1-5, the present invention provides a signaling device 10 that helps signal the imminent removal of a catheter or tube 11 from a patient's skull or other part of the body, such as an arm, leg or chest. As shown in FIGS. 1-3, generally, the device 10 includes a tube attachment mechanism 12, such as a clip that attaches to or is otherwise secured to the tube 11, a tether 14 and a patient attachment mechanism 16 that attaches to the patient's hair or skin. Therefore, if the patient attempts to pull the tube 11 out, as a result of the tethered connection between the tube attachment mechanism 12 and the patient attachment mechanism 16, the patient attachment mechanism 16 pulls on the hair or skin and signals or alerts the patient not to pull the tube out. In an exemplary embodiment, the patient feels a slight amount of pain or discomfort when the patient attachment mechanism 16 pulls on the hair or skin, which stops the patient from pulling on the tube 11. The tether 14 can be connected to the tube attachment mechanism 12 by any known method, for example, glue, adhesive, a clip, snap, Velcro, etc. In another embodiment, the tether 14 and tube attachment mechanism 12 can be unitary.

As shown in FIG. 1, in a preferred embodiment, the patient attachment mechanism 16 is a spring biased clip 15 that is secured to the patient's hair. As shown in FIG. 2, in another embodiment, the patient attachment mechanism is an adhesive patch 13 that is secured to the patient's skin. The adhesive patch 13 or some type of sticky gum or the like, can also be attached to the patient's hair. In another embodiment, patient attachment mechanism can include a hook or other sharp object that is attached to the skin and would hurt if removed or pulled on. As shown in FIG. 2, the adhesive patch 13 can include an eyelet in the center thereof to which the tether 14 is attached.

In a preferred embodiment, the tube attachment mechanism 12 provides temporary attachment to the tube. For example, the tube attachment mechanism 12 can be a spring biased clip, adhesive band or patch, elastic band or snap fitting plastic (or other pliable material) clip to which the tether 14 is connected. In another embodiment, the tube attachment mechanism 12 can be permanently attached to the tube 11. In another embodiment, the tether can be omitted and the tube attachment mechanism 12 can be connected directly to the patient attachment mechanism 16.

FIG. 3 shows the device 10 connected to a patient's hair. More particularly, FIG. 3 shows the tether 14 extending from the tube attachment mechanism 12 and the patient attachment mechanism 16 secured to the patient's hair. It also shows some slack between the tube attachment mechanism 12 and the point where the tube 11 enters the skull. With the tether 14 taught, as shown in FIG. 3, the device 10 can work effectively and will pull on the patient's hair or skin before any significant tension is placed on the portion of the tube 11 between the tube attachment mechanism 12 and the opening in the skull (i.e., the portion of the tube 11 distal to the tube attachment mechanism 12.

FIGS. 4-6 show another embodiment, of the present invention that includes a device 30 with a tube attachment mechanism 12 having a plurality of tethers 14 extending outwardly therefrom. Each of the tethers 14 include a patient attachment mechanism 16 at a distal end thereof that attaches to the patient's hair or skin.

In another embodiment, the device 30 can be designed to be more attractive or visually appealing to patients, such as children. For example, the device can be formed as an animal or other creature and the tethers 14 made to be legs with clips on the ends. FIGS. 5-6 show such an embodiment of the invention. In FIG. 5, the device 30 is formed or shaped like an octopus and includes clips 15 as the patient attachment mechanisms 16. In FIG. 6, the device 30 is formed or shaped like an octopus and includes adhesive patches 13 as the patient attachment mechanisms 16. The body portion of the octopus is the tube attachment mechanism 12 and the tentacles are the tethers 14. The opening in the octopus head and body can include elastic, adhesive or the like so that can be secured to the tube 11. In another embodiment, it can be permanently attached to and included with the tube 11 or catheter. An embodiment of the invention can include the method of allowing a patient to choose from a plurality of differently designed or colored devices 10 or 30.

The invention also includes the method of forming an opening in the patient's skull, inserting the tube into the opening and attaching the clip to the patient's hair or skin.

In another embodiment, the device 10 can include an audible or visual signal. Preferably, when the tether 14 is pulled, a circuit is closed, which causes the audible signal to be played. For example, the device 10 can include a slide tongue, such as the slide tongue taught in U.S. Pat. No. 8,341,858, the entirety of which is incorporated by reference herein.

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof, means any connection or coupling, either direct or indirect, between two or more elements; the coupling of connection between the elements can be physical, logical, or a combination thereof. Additionally, the words “herein,” “above,” “below,” and words of similar import, when used in this application, shall refer to this application as a whole and not to any particular portions of this application. Where the context permits, words in the above Detailed Description of the Preferred Embodiments using the singular or plural number may also include the plural or singular number respectively. The word “or” in reference to a list of two or more items, covers all of the following interpretations of the word: any of the items in the list, all of the items in the list, and any combination of the items in the list.

The above-detailed description of embodiments of the disclosure is not intended to be exhaustive or to limit the teachings to the precise form disclosed above. While specific embodiments of and examples for the disclosure are described above for illustrative purposes, various equivalent modifications are possible within the scope of the disclosure, as those skilled in the relevant art will recognize. For example, while processes or blocks are presented in a given order, alternative embodiments may perform routines having steps, or employ systems having blocks, in a different order, and some processes or blocks may be deleted, moved, added, subdivided, combined, and/or modified to provide alternative or subcombinations. Each of these processes or blocks may be implemented in a variety of different ways. Also, while processes or blocks are at times shown as being performed in series, these processes or blocks may instead be performed in parallel, or may be performed, at different times. Further any specific numbers noted herein are only examples: alternative implementations may employ differing values or ranges.

The teachings of the disclosure provided herein can be applied to other systems, not necessarily the system described above. The elements and acts of the various embodiments described above can be combined to provide further embodiments.

Any patents and applications and other references noted above, including any that may be listed in accompanying filing papers, are incorporated herein by reference in their entirety. Aspects of the disclosure can be modified, if necessary, to employ the systems, functions, and concepts of the various references described above to provide yet further embodiments of the disclosure.

These and other changes can be made to the disclosure in light of the above Detailed Description of the Preferred Embodiments. While the above description describes certain embodiments of the disclosure, and describes the best mode contemplated, no matter how detailed the above appears in text, the teachings can be practiced in many ways. Details of the system may vary considerably in its implementation details, while still being encompassed by the subject matter disclosed herein. As noted above, particular terminology used when describing certain features or aspects of the disclosure should not be taken to imply that the terminology is being redefined herein to be restricted to any specific characteristics, features or aspects of the disclosure with which that terminology is associated. In general, the terms used in the following claims should not be construed to limit the disclosures to the specific embodiments disclosed in the specification unless the above Detailed Description of the Preferred Embodiments section explicitly defines such terms. Accordingly, the actual scope of the disclosure encompasses not only the disclosed embodiments, but also all equivalent ways of practicing or implementing the disclosure under the claims.

Accordingly, although exemplary embodiments of the invention have been shown and described, it is to be understood that all the terms used herein are descriptive rather than limiting, and that many changes, modifications, and substitutions may be made by one having ordinary skill in the art without departing from the spirit and scope of the invention.

What is claimed is:

1. A device for signaling the imminent removal of a tube from an opening in a patient's skin, the device comprising:
a tube attachment mechanism, wherein the tube attachment mechanism is configured to be temporarily secured to a tube,
a tether having a proximal end and a distal end, wherein the proximal end of the tether is secured to the tube attachment mechanism, and
a patient attachment mechanism attached to the distal end of the tether.

2. The device of claim 1 wherein the patient attachment mechanism is a clip that is configured to be clipped to a patient's hair.

3. The device of claim 1 wherein the patient attachment mechanism is an adhesive patch configured to be adhered to a patient's skin.

4. The device of claim 1 further comprising a plurality of tethers each having a proximal end and a distal end, wherein the proximal end of each tether is secured to the tube attachment mechanism, and a patient attachment mechanism attached to the distal end of each of the tethers.

5. The device of claim 4 wherein each of the patient attachment mechanisms is a clip that is configured to be clipped to a patient's hair.

6. The device of claim 4 wherein each of the patient attachment mechanisms is an adhesive patch configured to be adhered to a patient's skin.

7. The device of claim 1 further comprising a tube, wherein the tube attachment mechanism is permanently attached to the tube.

8. The device of claim 1 further comprising a tube, wherein the tube and the device are provided in a kit.

9. A method comprising the steps of:
obtaining a tube,
obtaining a device for signaling the imminent removal of the tube from an opening in a patient's skin, the device comprising:
a tube attachment mechanism, wherein the tube attachment mechanism is configured to be temporarily secured to the tube,
a tether having a proximal end and a distal end, wherein the proximal end of the tether is secured to the tube attachment mechanism, and
a patient attachment mechanism attached to the distal end of the tether,
securing the tube attachment mechanism to the tube,
forming an opening in a patient's skin,
inserting the tube into the opening, and
securing the patient attachment mechanism to the patient.

10. The method of claim 9 wherein the patient attachment mechanism is secured to hair on the patient's head.

11. The method of claim 9 wherein the patient attachment mechanism is secured to the patient's skin.

12. The method of claim 9 wherein the device includes a plurality of tethers each having a proximal end and a distal end, wherein the proximal end of each tether is secured to the tube attachment mechanism, and a patient attachment mechanism attached to the distal end of each of the tethers, wherein the method further comprises the step of securing each of the patient attachment mechanisms to the patient.

13. The method of claim 12 wherein each of the patient attachment mechanisms is a clip.

14. The method of claim 13 wherein each of the patient attachment mechanisms is an adhesive patch.

15. The method of claim 9 wherein the tube is inserted into an opening in the patient's skin and an opening in the patient's skull.
